(19) United States
(12) Patent Application Publication
(10) Pub. No.: US 2020/0384278 A1
(43) Pub. Date: Dec. 10, 2020

(54) Method for Combination Cold Atmospheric Plasma and CAR-T Therapy

(71) Applicant: Jerome Canady Research Institute for Advanced Biological and Technological Sciences, Takoma Park, MD (US)

(72) Inventors: Jerome Canady, Lakeland, FL (US); Saravana Murthy, Owings Mills, MD (US); Xiaoqian Cheng, Fairfax, VA (US); Taisen Zhuang, Vienna, VA (US)

(21) Appl. No.: 16/892,651

(22) Filed: Jun. 4, 2020

Related U.S. Application Data
(60) Provisional application No. 62/857,022, filed on Jun. 4, 2019.

(51) Int. Cl. A61N 1/44; A61K 35/26; A61K 47/68

(52) U.S. Cl. CPC A61N 1/44; A61K 47/6803; A61K 47/6845; A61K 35/26

(57) ABSTRACT

A method for treatment of cancer using Cold Atmospheric Plasma treatment combined with CAR-T Therapy.

### CROSS-REFERENCE TO RELATED APPLICATIONS

The present application claims the benefit of the filing date of U.S. Provisional Patent Application Ser. No. 62/857,022 filed by the present inventors on Jun. 4, 2019.

The aforementioned provisional patent application is hereby incorporated by reference in its entirety.

### STATEMENT REGARDING FEDERALLY SPONSORED RESEARCH OR DEVELOPMENT

None.

### BACKGROUND OF THE INVENTION

### Field of the Invention

The present invention relates to systems and methods for treating cancer with cold atmospheric plasma.

### Brief Description of the Related Art

Recent progress in atmospheric plasmas led to creation of cold plasmas with ion temperatures close to room temperature. Cold non-thermal atmospheric plasmas can have tremendous applications in biomedical technology. K. H. Becker, K. H. Shoenbach and J. G. Eden “Microplasma and applications” J. Phys. D.: Appl. Phys. 39, R55-R70 (2006). In particular, plasma treatment can potentially offer a minimum-invasive surgery that allows specific cell removal without influencing the whole tissue. Conventional laser surgery is based on thermal interaction and leads to accidental cell death i.e. necrosis and may cause permanent tissue damage. In contrast, non-thermal plasma interaction with tissue may allow specific cell removal without necrosis. In particular, these interactions include cell detachment without affecting cell viability, controllable cell death etc. It can be used also for cosmetic methods of regenerating the reticular architecture of the dermis. The aim of plasma interaction with tissue is not to denaturate the tissue but rather to operate under the threshold of thermal damage and to induce chemically specific response or modification. In particular presence of the plasma can promote chemical reaction that would have desired effect. Chemical reaction can be promoted by tuning the pressure, gas composition and energy. Thus, the important issues are to find conditions that produce effect on tissue without thermal treatment. Overall plasma treatment offers the advantage that—can never be thought of in most advanced laser surgery. E. Stoffels, I. E Kieft, R. E. J Sladek, L. J. M van den Bedem, E. P van der Laan, M. Steinbuch “Plasma needle for in vivo medical treatment: recent developments and perspectives” Plasma Sources Sci. Technol. 15, S169-S180 (2006).

Several different systems and methods for performing Cold Atmospheric Plasma (CAP) treatment have been disclosed. For example, U.S. Published Patent Application No. 2014/0378892 discloses a two-electrode system for CAP treatment. U.S. Pat. No. 9,999,462 discloses a converter unit for using a traditional electrosurgical system with a single electrode CAP accessory to perform CAP treatment.

As a near-room temperature ionized gas, cold atmospheric plasma (CAP) has demonstrated its promising capability in cancer treatment by causing the selective death of cancer cells in vitro. See, Yan D, Sherman J H and Keidar M, “Cold atmospheric plasma, a novel promising anti-cancer treatment modality,” Oncotarget. 8 15977-15995 (2017); Keidar M, “Plasma for cancer treatment,” Plasma Sources Sci. Technol. 24 33001 (2015); Hirst A M, Frame F M, Arya M, Maitland N J and O'Connell D, “Low temperature plasmas as emerging cancer therapeutics: the state of play and thoughts for the future,” Tumor Biol. 37 7021-7031 (2016). The CAP treatment on several subcutaneous xenograft tumors and melanoma in mice has also demonstrated its potential clinical application. See, Keidar M, Walk R, Shashurin A, Srinivasan P, Sandler A, Dasgupta S, Ravi R, Guerrero-Preston R and Trink B, “Cold plasma selectivity and the possibility of a paradigm shift in cancer therapy,” Br. J. Cancer. 105 1295-301 (2011); Vandamme M, Robert E, Dozias S, Sobilo J, Lerondel S, Le Pape A and Pouvesle J-M, “Response of human glioma U87 xenografted on mice to non thermal plasma treatment,” Plasma Med. 1 27-43 (2011); Brulle L, Vandamme M, Ries D, Martel E, Robert E, Lerondel S, Trichet V, Richard S, Pouvesle J M and Le Pape A, “Effects of a Non thermal plasma treatment alone or in combination with gemcitabine in a MIA PaCa2-luc orthotopic pancreatic carcinoma model,” PLoS One. 7 e52653 (2012); and Chernets N, Kurpad D S, Alexeev V, Rodrigues D B and Freeman T A, “Reaction chemistry generated by nanosecond pulsed dielectric barrier discharge treatment is responsible for the tumor eradication in the B16 melanoma mouse model,” Plasma Process. Polym. 12 1400-1409 (2015).

The rise of intracellular reactive oxygen species (ROS), DNA damage, mitochondrial damage, as well as apoptosis have been extensively observed in the CAP-treated cancer cell lines. See, Ahn H J, Kim K Il, Kim G, Moon E, Yang S S and Lee J S, “Atmospheric-pressure plasma jet induces apoptosis involving mitochondria via generation of free radicals,”. PLoS One. 6 e28154 (2011); Ja Kim S, Min Joh H and Chung T H, “Production of intracellular reactive oxygen species and change of cell viability induced by atmospheric pressure plasma in normal and cancer cells,” Appl. Phys. Lett. 103 153705 (2013); and Yan D, Talbot A, Nourmohammadi N, Sherman J H, Cheng X and Keidar M, “Toward understanding the selective anticancer capacity of cold atmospheric plasma—a model based on aquaporins (Review),” Biointerphases. 10 040801 (2015). The increase of intracellular ROS may be due to the complicated intracellular pathways or the diffusion of extracellular ROS through the cellular membrane. See, Yan D, Xiao H, Zhu W, Nourmohammadi N, Zhang L G, Bian K and Keidar M, “The role of aquaporins in the anti-glioblastoma capacity of the cold plasma-stimulated medium,” J. Phys. D. Appl. Phys. 50 055401 (2017). However, the exact underlying mechanism is still far from clear.

Cancer cells have shown specific vulnerabilities to CAP. See, Yan D, Talbot A, Nourmohammadi N, Cheng X, Canady J, Sherman J and Keidar M, “Principles of using cold atmospheric plasma stimulated media for cancer treatment,” Sci. Rep. 5 18339 (2015)

Understanding the vulnerability of cancer cells to CAP will provide key guidelines for its application in cancer treatment. Only two general trends about the cancer cells' vulnerability to CAP treatment have been observed in vitro based on just a few cell lines. First, one study just compared the cytotoxicity of CAP treatment on the cancer cell lines expressing p53 with the same treatment on the cancer cell lines without expressing p53. The cancer cells expressing the p53 gene were shown to be more resistant to CAP treatment than p53 minus cancer cells. Ma Y, Ha C S, Hwang S W, Lee H J, Kim G C, Lee K W and Song K, “Non-thermal atmospheric pressure plasma preferentially induces apoptosis in p53-mutated cancer cells by activating ROS stress-response pathways,” PLoS One. 9 e91947 (2014). p53, a key tumor suppressor gene, not only restricts abnormal cells via the induction of growth arrest or apoptosis, but also protects the genome from the oxidative damage of ROS such as H₂O₂ through regulating the intracellular redox state. Sablina A A, Budanov A V, Ilyinskaya G V, Larissa S, Kravchenko J E and Chumakov P M, “The antioxidant function of the p53 tumor suppressor,” Nat. Med. 11 1306 (2005). p53 is an upstream regulator of the expression of many anti-oxidant enzymes such as glutathione peroxidase (GPX), glutaredoxin 3 (Grx3), and manganese superoxide dismutase (MnSOD). Maillet A and Pervaiz S, “Redox regulation of p53, redox effectors regulated by p53: a subtle balance,” Antioxid. Redox Signal. 16 1285-1294 (2012). In addition, the cancer cells with a lower proliferation rate are more resistant to CAP than cancer cells with a higher proliferation rate. Naciri M, Dowling D and Al-Rubeai M, “Differential sensitivity of mammalian cell lines to non-thermal atmospheric plasma,” Plasma Process. Polym. 11 391-400 (2014). This trend may be due to the general observation that the loss of p53 is a key step during tumorigenesis. Tumors at a high tumorigenic stage are more likely to have lost p53. See, Fearon E F and Vogelstein B, “A genetic model for colorectal tumorigenesis,” Cell. 61 759-767 (1990).

Despite the complicated interaction between CAP and cancer cells, the initial several hours after treatment has been found to be an important stage for the cytotoxicity of CAP. The anti-cancer ROS molecules in the extracellular medium are completely consumed by cells during this time period. After the initial several hours, replacing the medium surrounding the cancer cells does not change the cytotoxicity of CAP. See, Yan D, Cui H, Zhu W, Nourmohammadi N, Milberg J, Zhang L G, Sherman J H and Keidar M, “The specific vulnerabilities of cancer cells to the cold atmospheric plasma-stimulated solutions,” Sci. Rep. 7 4479 (2017).

Allogeneic or autologous chimeric antigen receptor (CAR) therapy has been the subject of many recent studies. In this therapy, T cells are collected from the patient or donor by apheresis, and the T cells are then expanded and genetically modified using one of several approaches. The CAR-T cells then are infused into the patient. In one prior study, a large panel of single-chain variable fragments of an antibody that bind to CD70 were generated and formatted into CARs. Anti-CD70 allogeneic CAR-T cells were identified, selected and studied in short- and long-term cytotoxicity assays, which confirmed their ability to kill renal cell carcinoma (RCC) cells in vitro and in multiple in vivo models. Anti-CD70 AlloCAR T therapy candidates were ranked based on tonic signaling, transduction efficiency, phenotype, activation status and expansion. The preclinical study also found that anti-CD70 AlloCAR T cells could be successfully manufactured in a large-scale process. See, “Allogene Therapeutics Presents Preclinical Data Demonstrating the Potential of AlloCAR T™ Therapy in Renal Cell Carcinoma (RCC) at the 2019 AACR Annual Meeting,” Apr. 1, 2019. See also, U.S. Published Patent Application No. 20120288512 entitled “Anti-CD70 Antibody-Drug Conjugates and Their Use for the Treatment of Cancer and Immune Disorders,” U.S. Published Patent Application No. 20180230224 entitled “CD70 BINDING MOLECULES AND METHODS OF USE THEREOF” and U.S. Published Patent Application No. 20180208671 entitled “ANTI-CD70 CHIMERIC ANTIGEN RECEPTORS,” all of which are hereby incorporated by referenced in their entirety.

### SUMMARY OF THE INVENTION

In a preferred embodiment, the present invention is a treatment approach for cancer using Cold Atmospheric Plasma combined with CAR-T therapy. In a preferred embodiment, a patient with RCC is prepared for CAR-T CD70 therapy by performing surgery and resecting the RCC tumor. The periphery of the resected area in the patient is then treated with cold atmospheric plasma (CAP). The CAP induces metabollic suppression in only the remaining tumor cells in the patient and enhances the patient's response to drugs. The CAR-T CD70 cell therapy is then performed on the patient. The CAR-T CD70 therapy may comprise administering to the subject, in an amount effective for the treatment, an antibody-drug conjugate comprising an antibody that binds CD70 and is conjugated to a cytotoxic agent, wherein the antibody-drug conjugate is internalized into immune cells that express CD70, where it exerts a therapeutic effect.

Still other aspects, features, and advantages of the present invention are readily apparent from the following detailed description, simply by illustrating a preferable embodiments and implementations. The present invention is also capable of other and different embodiments and its several details can be modified in various obvious respects, all without departing from the spirit and scope of the present invention. Accordingly, the drawings and descriptions are to be regarded as illustrative in nature, and not as restrictive. Additional objects and advantages of the invention will be set forth in part in the description which follows and in part will be obvious from the description or may be learned by practice of the invention.

### BRIEF DESCRIPTION OF THE DRAWINGS

For a more complete understanding of the present invention and the advantages thereof, reference is now made to the following description and the accompanying drawings, in which:

FIG. 1A is a diagram illustrating a procedure for performing a cold atmospheric plasma treatment of cancer cells in combination with Allogeneic CAR-T CD70 therapy.

FIG. 1B is a flow chart illustrating steps of a procedure for performing a cold atmospheric plasma treatment of cancer cells in combination with Allogeneic CAR-T CD70 therapy.

FIG. 2A is a perspective view of a preferred embodiment of a gas-enhanced electrosurgical generator that may be used in a preferred embodiment of the present invention.

FIG. 2B is a block diagram of a cold atmospheric plasma generator in accordance with a preferred embodiment of the present invention.

FIG. 3A is a block diagram of an embodiment of a cold atmospheric plasma system with an electrosurgical generator and a low frequency converter for producing cold plasma.

FIG. 3B is a block diagram of an embodiment of an integrated cold atmospheric plasma system that can perform multiple types of plasma surgeries.

FIG. 4A is a block diagram of a preferred embodiment of pressure control system of a gas-enhanced electrosurgical generator in accordance with the present invention configured to perform an argon-enhanced electrosurgical procedure.

FIG. 4B is a block diagram of a preferred embodiment of pressure control system of a gas-enhanced electrosurgical generator in accordance with the present invention configured to perform a cold atmospheric plasma procedure.

FIG. 5 is perspective view of a cold atmospheric plasma probe that may be used in a preferred embodiment of the present invention.

FIG. 6A is an assembly view of a handpiece of a cold atmospheric plasma probe that may be used in a preferred embodiment of the present invention.

FIG. 6B is an assembly view of a cable harness of a cold atmospheric plasma probe that may be used in a preferred embodiment of the present invention.

### DETAILED DESCRIPTION OF THE PREFERRED EMBODIMENTS

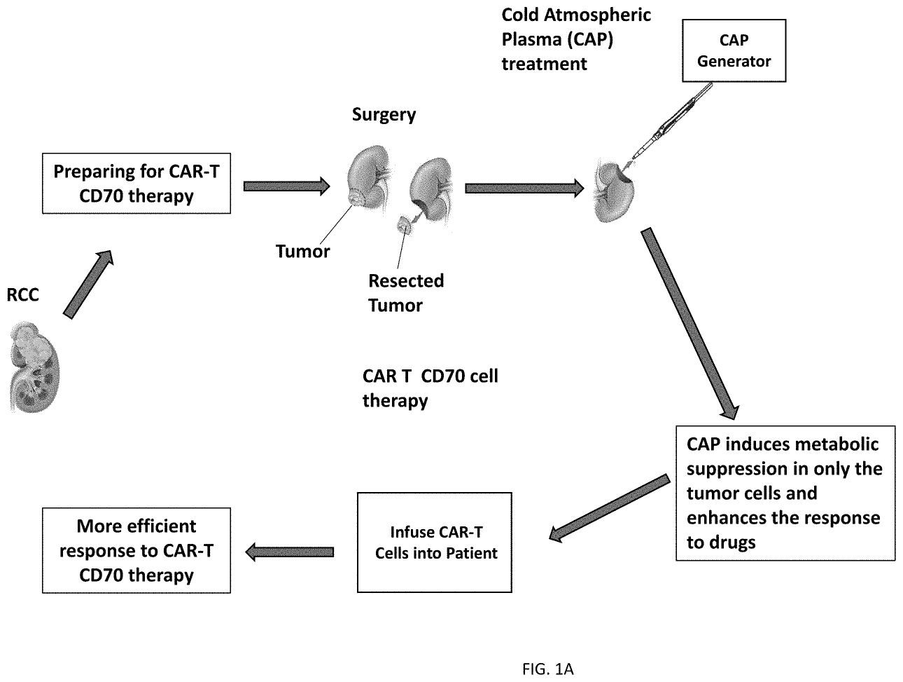

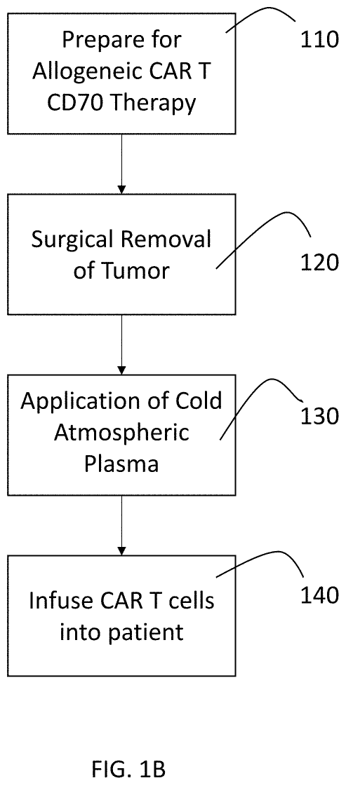

A preferred embodiment of the present invention is described with reference to FIG. 1A. A human organ with renal cell carcinoma (RCC) is prepared for CART-T CD70 therapy. The RCC tumor is identified and resected. The margins or periphery of the resected area of the organ are then treated with cold atmospheric plasma (CAP). The CAP treatment induces metabolic suppression in only the remaining tumor cells and enhances the response of the patient to medications or other treatments performed after the application of CAP. The CAR-T CD70 cell therapy is then performed by infusing the CAR-T cells into the patient. As described in U.S. Published Patent Application No. 20120288512, the CAR-T CD70 therapy may comprise administering to the patient, in an amount effective for the treatment, an antibody-drug conjugate comprising an antibody that binds CD70 and is conjugated to a cytotoxic agent, wherein the antibody-drug conjugate is internalized into immune cells that express CD70, where it exerts a therapeutic effect The combination of the CAR-T CD70 therapy with the CAP treatment provides for a more efficient and effective response of the patient to the CAR-T therapy.

Thus, as shown in FIG. 1B, a method can be performed in which cold atmospheric plasma treatment of cancer cells is performed in combination with Allogeneic CAR-T CD70 therapy. The patient is prepared for Allogeneic CAR-T CD70 therapy 110. Surgery is performed to remove a tumor in the patient 120. Cold Atmospheric Plasma (CAP) treatment is applied to the tissue surrounding the resected tumor 130. The CAP treatment induces metabolic suppression in only the tumor cells and enhances the response to the drugs. CAR-T cells are then infused into the patient 140. See, “AlloCAR T™ TARGETING CD70 FOR THE TREATMENT OF RENAL CELL CARCINOMA,” S. Panowski, et al., Allogene Therapeutics (April 2019), a copy of which is appended hereto and is hereby incorporated by reference in its entirety. A more efficient response to the CAR-T CD70 therapy is produced.

An exemplary CAR-T therapy that may be used in the present invention is described in S. Panowski, et al., “AlloCART™ TARGETING CD70 FOR THE TREATMENT OF RENAL CELL CARCINOMA.” Renal Cell Carcinoma (RCC) is a highly T-cell infiltrated tumor type with responsiveness to immuno-oncology agents. T cells can be genetically modified to express chimeric antigen receptors (CARs). To translate this approach for RCC treatment, expression data were mined and CD70 was identified as an antigen expressed in a high proportion of patients with RCC, with limited normal tissue expression on a fraction of activated lymphocytes and dendritic cells. Since CD70 expression is present on activated T cells, targeting it with a CAR could lead to fratricide and T cell exhaustion. Screens were specifically designed to identify CARs that were less impacted by these issues. A large panel of scFvs that bind to CD70 were generated and formatted into CARs. CD70 CAR-T cells were ranked based on tonic signaling, transduction efficiency, phenotype, activation status and expansion. A subset of CD70 CAR-T cells were moved into in vitro short and long-term cytotoxicity assays. Target cells expressing high, medium, and low levels of CD70 were utilized. CART cells were evaluated in vivo and robust anti-tumor activity was observed. Some candidates performed better with CD70 knockout and some worked irrespective of knockout. A cynomolgus monkey toxicity study was conducted with one clone formatted as a CD70-CD3 bispecific antibody and no unexpected findings were observed. Multiple off-switch CAR formats were evaluated. CD70 CAR-T cells were also successfully manufactured in a large-scale process. In summary, multiple CD70 CAR-T cells have been profiled and a subset selected for further investigation as potential clinical candidates.

The present invention may be used with other CAR-T therapies. Additionally, use of the CAP treatment may allow for reduced dosage for the CAR-T therapy as well as reduced dosages in chemotherapy and other treatments.

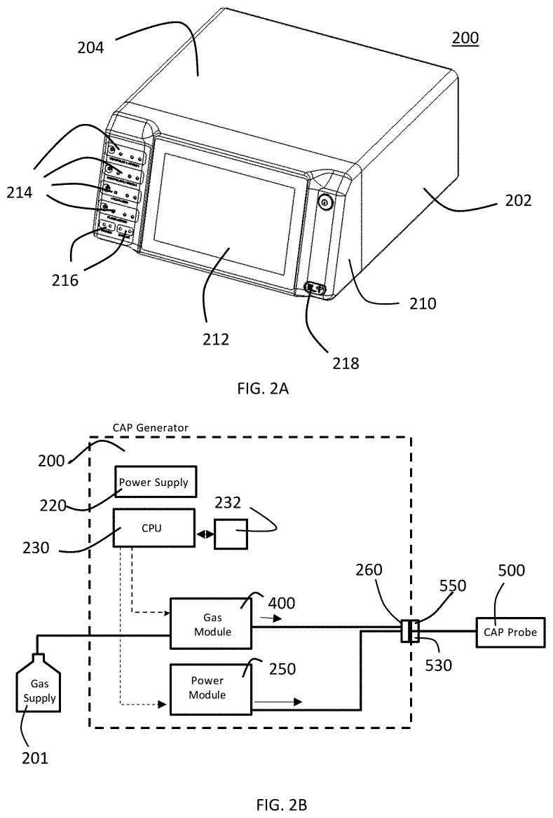

A preferred embodiment of a CAP enabled generator is described with reference to the drawings. A gas-enhanced electrosurgical generator 200 in accordance with a preferred embodiment of the present invention is shown in FIGS. 2A-2B. The gas-enhanced generator has a housing 202 made of a sturdy material such as plastic or metal similar to materials used for housings of conventional electrosurgical generators. The housing 202 has a removable cover 204. The housing 202 and cover 204 have means, such as screws, tongue and groove, or other structure for removably securing the cover to the housing. The cover 204 may comprise just the top of the housing or multiple sides, such as the top, right side and left side, of the housing 202. The housing 202 may have a plurality of feet or legs (not shown) attached to the bottom of the housing. The bottom of the housing 202 may have a plurality of vents (not shown) for venting from the interior of the gas-enhanced generator.

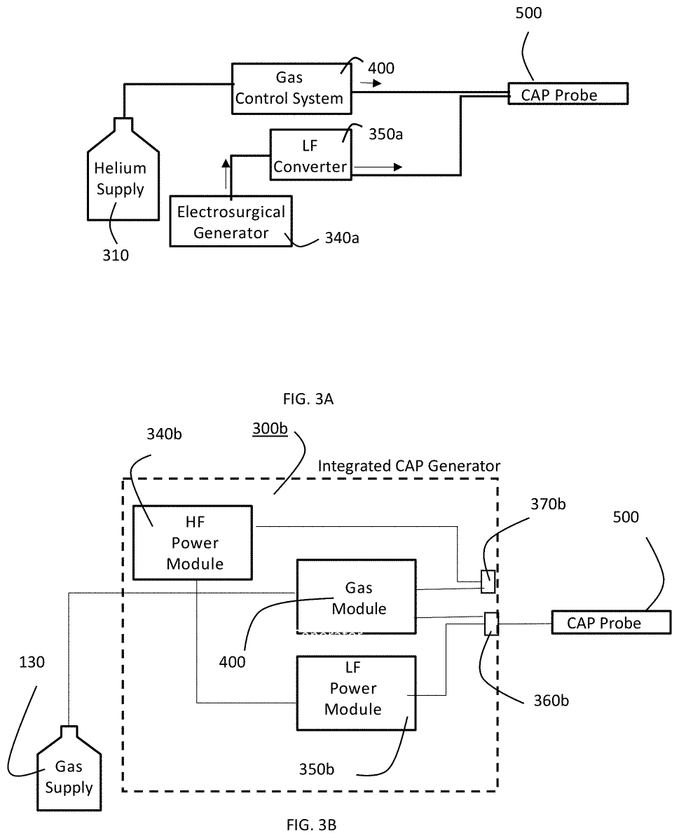

A generator housing front panel 210 is connected to the housing 202. On the face front panel 210 there is a touch-screen display 212 and there may be one or a plurality of connectors 214 for connecting various accessories to the generator 200. For a cold atmospheric plasma generator such as is shown in FIG. 2B, for example, there is a connector 260 for connecting a cold atmospheric probe 500. An integrated multi-function electrosurgical generator, such as is shown in FIG. 3B the plurality of connectors may include an argon plasma probe, a hybrid plasma probe, a cold atmospheric plasma probe, or any other electrosurgical attachment. The face of the front panel 210 is at an angle other than 90 degrees with respect to the top and bottom of the housing to provide for easier viewing and use of the touch screen display 212 by a user.

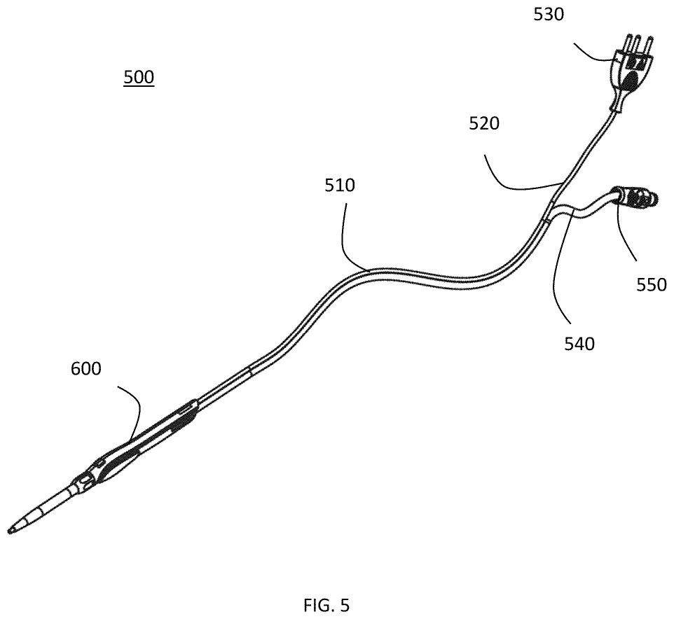

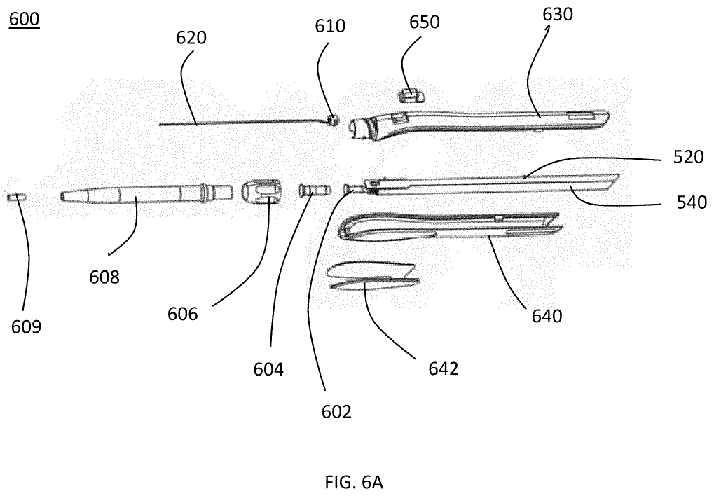

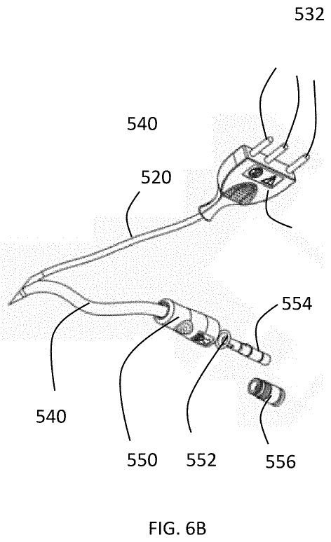

As shown in FIG. 2B, an exemplary cold atmospheric plasma (CAP) generator 200 has a power supply 220, a CPU (or processor or FPGA) 230 and a memory or storage 232. The system further has a display 212 (FIG. 2A), which may be the display of a tablet computer. The CPU 230 controls the system and receives input from a user through a graphical user interface displayed on display 212. The CAP generator further has a gas control module 400 connected to a source 201 of a CAP carrier gas such as helium. The CAP generator 200 further has a power module 250 for generating low frequency radio frequency (RF) energy, such as is described in U.S. Pat. No. 9,999,462, which is hereby incorporated by reference in its entirety. The power module 250 contains conventional electronics and/or transformers such as are known to provide RF power in electrosurgical generators. The power module 250 operates with a frequency between 10-200 kHz, which is referred to herein as a “low frequency,” and output peak voltage from 3 kV to 6 kV and preferably at a frequency near (within 20%) of 40 Hz, 100 Hz or 200 Hz. The gas module 400 and power module 250 are connected to connector 260 that allows for attachment of a CAP applicator 500 (as shown in FIGS. 5, 6A and 6B) to be connected to the generator 200 via a connector having an electrical connector 530 and gas connector 550.

As shown in FIG. 3B, other arrangements for delivery of the carrier gas and the electrical energy may be used with the invention. In FIG. 3B, an integrated CAP generator 300b is connected to a source 310 of a carrier gas (helium in this example), which is provided to a gas control system 400, which supplies the gas at a controlled flow rate to CAP applicator 500. A high frequency (HF) power module 340b supplies high frequency (HF) energy to a low frequency power module (converter) 350b, which outputs electrical energy having a frequency in the range of 10 kHz to 200 kHz and an output voltage in the range of 3 kV to 6 Kv. This type of integrated generator will have both a CAP connector 360b for connecting a CAP applicator or other CAP accessory and a connector 370b for attaching HF electrosurgical attachments such as an argon plasma or hybrid plasma probe (not shown).

Another embodiment, shown in FIG. 3A, has a carrier gas source 310 connected to a conventional gas control system 370, which in turn is connected to the CAP applicator 500, and a conventional electrosurgical generator 340 connected to a low frequency (LF) converter 350a, which is then connected to the CAP probe 500.

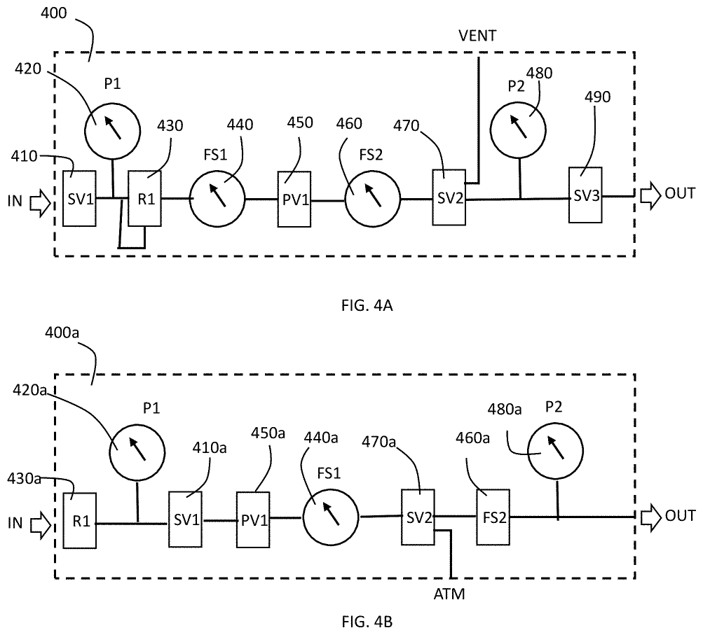

FIG. 4A is a schematic flow diagram illustrating the gas flow through the gas control module 400 and the method by which the module 400 controls the gas flow in accordance with a preferred embodiment of the present invention. As shown in FIG. 4A, the gas enters the gas control module at an inlet port (IN) 401 and proceeds to first solenoid valve (SV1) 410, which is an on/off valve. In an exemplary embodiment, the gas enters the gas module at a pressure of 75 psi. The gas then proceeds to a first pressure sensor (P1) 420, to a first pressure regulator (R1) 430. In an exemplary embodiment, the first pressure regulator (R1) 430 reduces the pressure of the gas from 75 psi to 18 psi. After the pressure regulator (R1) 430, the gas proceeds to flow sensor (FS1) 440, which senses the flow rate of the gas. Next, the gas proceeds to proportional valve (PV1) 450, which permits adjustment of a percentage of the opening in the valve. The gas then proceeds to a second flow sensor (FS2) 460, which senses the flow rate of the gas. This second flow sensor (FS2) 460 provides redundancy and thus provides greater safety and accuracy in the system. Next the gas proceeds to a second solenoid valve (SV2) 470, which is a three-way valve that provides for a vent function that can allow gas to exit the module through a vent 472. The gas then proceeds to a second pressure sensor (P2) 480, which provides a redundant pressure sensing function that again produces greater safety and accuracy of the system. Finally, the gas proceeds to a third solenoid valve (SV3) 490, which is a two-way on/off valve that is normally closed and is the final output valve in the module. The gas exits the module at an output port (OUT) 499, which is connected to tubing or other channel that provides a passageway for the gas to flow to an accessory connected to the electrosurgical unit.

FIG. 4B is a schematic flow diagram of an alternate embodiment of a gas control module illustrating the gas flow through the gas control module 400a and the method by which the module 400a controls the gas flow in accordance with a preferred embodiment of the present invention. As shown in FIG. 4B, the gas enters the gas control module at an inlet port 401a and proceeds to a first pressure regulator (R1) 430a. In an exemplary embodiment, the first pressure regulator (R1) 430a reduces the pressure of the gas from about 50-100 psi to 15-25 psi. After the pressure regulator (R1) 430a, the gas proceeds to a first pressure sensor (P1) 420a and then to a first solenoid valve (SV1) 410a, which is an on/off valve. Next, the gas proceeds to proportional valve (PV1) 450a, which permits adjustment of a percentage of the opening in the valve. Next, the gas proceeds to flow sensor (FS1) 440a, which senses the flow rate of the gas. Next the gas proceeds to a second solenoid valve (SV2) 470a, which is a three-way valve that provides for a vent function that can allow gas to exit the module through a vent 472a. The gas then proceeds to a second flow sensor (FS2) 460a, which senses the flow rate of the gas. This second flow sensor (FS2) 460a provides redundancy and thus provides greater safety and accuracy in the system. The gas then proceeds to a second pressure sensor (P2) 480a, which provides a redundant pressure sensing function that again produces greater safety and accuracy of the system. The gas exits the module at and output port 499a, which is connected to tubing or other channel that provides a passageway for the gas to flow to an accessory connected to the electrosurgical unit.

The various valves and sensors in either embodiment of the module are electrically connected to a main PCB Board through a connector. The PCB connector is connected to a PCB Board that has a microcontroller (such as CPU). As previously noted, a plurality of gas modules can be in a single gas control unit or single electrosurgical generator to provide control of multiple differing gases. The plurality of gas control modules further may be connected to the same PCB Board, thus providing common control of the modules.

In the above-disclosed embodiment, a cold atmospheric plasma below 35° C. is produced. When applied to the tissue surrounding the surgical area, the cold atmospheric plasma induces metabolic suppression in only the tumor cells and enhances the response to the drugs that are injected into the patient.

The cold plasma applicator 500 may be in a form such as is disclosed in U.S. Pat. No. 10,405,913 and shown in FIGS. 5, 6A and 6B. A hand piece assembly 600 has a top side piece 630 and a bottom side piece 640. A control button 650 extends from the interior of the hand piece through an opening in the top side piece 630. Within the hand piece 600 is body connector funnel 602, PCB board 608, electrical wiring 520 and hose tubing (PVC medical grade) 540. The wiring 520 and hose tubing 540 are connected to one another to form a wire and tubing bundle 510. A grip over mold 642 extends over the bottom piece portion 640. In other embodiments, a grip may be attached to the bottom piece 640 in other manners. A probe or scalpel assembly is attached to the end of the hand piece. The probe assembly has non-bendable telescoping tubing 606, a ceramic tip 609, a column nut or collet 606 and body connector tubing 604. The hose tubing 540 extends out of the proximal end of the hand piece to a body gas connector 550, which has an O-ring 552, gas connector core 554 and gas connector tip 556 for connecting to a connector on a gas-enhanced electrosurgical generator. The printed circuit board 608 connects to electrical wiring 520 which leads to electrical connector 530 having electrical pins 532. Inside the handpiece 600 is an electrode 620 and conductive connector 610. There is a control button 650 for controlling the application of electrical energy.

While the present application discloses a specific type of cold plasma, other types of plasma jets may be used in the present invention.

The foregoing description of the preferred embodiment of the invention has been presented for purposes of illustration and description. It is not intended to be exhaustive or to limit the invention to the precise form disclosed, and modifications and variations are possible in light of the above teachings or may be acquired from practice of the invention. The embodiment was chosen and described in order to explain the principles of the invention and its practical application to enable one skilled in the art to utilize the invention in various embodiments as are suited to the particular use contemplated. It is intended that the scope of the invention be defined by the claims appended hereto, and their equivalents. The entirety of each of the aforementioned documents is incorporated by reference herein.

What is claimed is:

1. A method for applying cold atmospheric plasma treatment to cancer cells in combination with allogeneic CAR-T therapy.

2. The method according to claim 1 wherein said CAR-T therapy comprises allogeneic CAR-T CD70 therapy.

3. The method according to claim 2 wherein said step of infusing CAR-T cells into the patient comprises:
administering to the subject, in an amount effective for the treatment, an antibody-drug conjugate comprising an antibody that binds CD70 and is conjugated to a cytotoxic agent, wherein the antibody-drug conjugate is internalized into immune cells that express CD70, where it exerts a therapeutic effect.

4. method for treating renal cell carcinoma in a patient comprising the steps of:
surgically removing a renal cell carcinoma tumor from a surgical area;
treating margins of the surgical area with cold atmospheric plasma; and
infusing CAR-T cells into the patient.

5. The method according to claim 4 wherein said CAR-T therapy comprises allogeneic CAR-T CD70 therapy.

6. The method according to claim 5 wherein said step of infusing CAR-T cells into the patient comprises:
administering to the subject, in an amount effective for the treatment, an antibody-drug conjugate comprising an antibody that binds CD70 and is conjugated to a cytotoxic agent, wherein the antibody-drug conjugate is internalized into immune cells that express CD70, where it exerts a therapeutic effect.
